(19) United States
(12) Patent Application Publication
(10) Pub. No.: US 2026/0224870 A1
(43) Pub. Date: Aug. 6, 2026

(54) Application Device for Microneedles, System for Microneedle Application, and Method for Providing a System for Microneedle Application

(71) Applicant: LTS Lohmann Therapie-Systeme AG, Andernach (DE)

(72) Inventors: Miriam Britten, Andernach (DE); Maral Heidary Dastjerdi, Remagen (DE); Sebastian Scherr, Neuhäusel (DE)

(21) Appl. No.: 19/150,953

(22) PCT Filed: Dec. 28, 2023
(86) PCT No.: PCT/EP2023/087922

§ 371 Date: Jul. 25, 2025

(30) Foreign Application Priority Data
Jan. 27, 2023 (DE) 10 2023 101 977.5

(51) Int. Cl. A61M 37/00

(52) U.S. Cl. CPC A61M 37/0015; A61M 2037/0023; A61M 2037/0061; A61M 2205/02

(57) ABSTRACT

An application device for microneedles including a microneedle receptacle, a reservoir receptacle for receiving a reservoir, and a contact barrier for spacing a microneedle array-received in the microneedle receptacle and a reservoir received in the reservoir receptacle. Further, a system for microneedle application and a method for providing a system for microneedle application.

### CROSS-REFERENCE TO RELATED APPLICATIONS

This application is the United States national phase of International Patent Application No. PCT/EP2023/087922 filed Dec. 28, 2023, and claims priority to Ger-man Patent Application No. 10 2023 101 977.5 filed Jan. 27, 2023, the disclosures of which are hereby incorporated by reference in their entireties.

### BACKGROUND OF THE INVENTION

### Field of the Invention

The invention relates to an application device for microneedles, a system for microneedle application, and a method for providing a system for microneedle application.

### Description of Related Art

Beside classic forms of active ingredient administration, such as for example swallowing medications or injecting medicine or the like, there are concepts for active ingredient application via the skin. In this regard, microneedles are of particular importance.

Microneedle arrays, also referred to as microarrays, have a plurality of microneedles that are typically arranged on a support element, such as a patch, a plaster or the like, or connected to a support element. Such microneedle arrays include a high number of microneedles, for example 100 to 600 needles per cm². In particular, the needles have a short length, so that when the microneedles are pressed into a patient's skin, the needles penetrate the skin only so far that nerves and blood vessels make no contact with needle tips, if possible. The microneedle arrays comprise an active ingredient or a medication. For example, the corresponding active ingredient may be provided on a surface of the needles or may be arranged in the needles. The needles can be made of a material dissolving in the skin, for example.

There are also approaches to hydrogel microneedle arrays, as described e.g. in Donnelly RF, McCrudden MTC, Zaid Alkilani A, Larrañeta E, McAlister E, et al. “Hydrogel-Forming Microneedles Prepared from ”Super Swelling“ Polymers Combined with Lyophilized Wafers for Transdermal Drug Delivery” (2014) PLOS ONE 9(10). These hydrogel microneedle arrays have white swelling microneedles and generally no active ingredient. Instead, after application, they absorb fluids, in particular the interstitial fluid of the skin, and form a connection, in particular a continuous and/or unblocked connection, between the skin, preferably the dermal microcirculation, and a drug reservoir connected to the microneedles. Generally, such hydrogel microneedle arrays initially serve as a tool for penetrating the stratum corneum barrier. After application, they also function in particular as a speed-controlling dispensing device. The speed is controlled, for example, by adjusting the needles, which serve as a membrane.

There are numerous problems with current concepts for hydrogel microneedle arrays. Depending on the moisture content of the reservoir, this can lead to diffusion of the water and/or other solvents contained in the microneedle array. This can result, for example, in the microneedles swelling before application and/or at least becoming so soft that the necessary penetration of the skin, in particular the stratum corneum, can no longer be ensured.

### SUMMARY OF THE INVENTION

The object of the invention is to provide an application device for microneedles, a system for microneedle application, and a method for providing a system for microneedle application, wherein the handling, in particular the storage, of microneedle arrays is improved.

According to the invention, the object is achieved by an application device for microneedles, a system for microneedle application, and a method for providing a system for microneedle application as described herein.

The application device for microneedles according to the invention is in particular an application device for hydrogel microneedles. Preferably it is an application device for a microneedle array, particularly preferably a hydrogel microneedle array. The application device has a microneedle receptacle, in particular for receiving a microneedle array. Furthermore, the application device has a reservoir receptacle for receiving a reservoir, in particular one containing an active ingredient. The application device, in particular the microneedle receptacle and the reservoir receptacle, is preferably configured to receive the microneedle array and the reservoir opposite each other, preferably on the end face. Furthermore, the application device has a contact barrier for spacing a microneedle array received in the microneedle receptacle and a reservoir received in the reservoir receptacle. In particular, the contact barrier is a diffusion barrier. The application device preferably comprises a housing, in particular made of plastic. The microneedle receptacle and/or the reservoir receptacle is preferably designed in one piece, also referred to as integral, with the housing. It is preferable that the microneedle receptacle and/or the reservoir receptacle and the housing are made of a common material, in particular one that merges into the other. Preferably, the microneedle receptacle and/or the reservoir receptacle is formed as an opening in the housing. The contact barrier is particularly advantageous in preventing diffusion of the contained water and/or another solvent into the microneedle array.

In a preferred embodiment, the contact barrier is a gap, in particular an air gap (28). The gap, in particular the air gap (28), is preferably formed between a microneedle array received in the microneedle receptacle and a reservoir received in the reservoir receptacle and/or between the microneedle receptacle and the reservoir receptacle.

In a preferred embodiment, the gap formed by the contact barrier, preferably between a microneedle array received in the microneedle receptacle and a reservoir received in the reservoir receptacle and/or between the microneedle receptacle and the reservoir receptacle, is 0.01 mm to 3 mm, preferably 0.05 mm to 1 mm, particularly preferably 0.1 mm to 0.5 mm.

In a preferred embodiment, the application device, preferably the contact barrier, is configured such that the contact barrier can be overcome by a, in particular force-actuated, relative movement of a microneedle array received in the microneedle receptacle relative to a reservoir received in the reservoir receptacle. The force-actuated relative movement is in particular a contact pressure, preferably a manual contact pressure, of a user. In particular, a gap must be overcome by the relative movement.

In a preferred embodiment, the application device has an intermediate element, in particular a spacer, between the microneedle receptacle and the reservoir receptacle to form the contact barrier. In particular, the intermediate element is a web.

In a preferred embodiment, the intermediate element has an opening between the microneedle receptacle and the reservoir receptacle, in particular to form a connection between the microneedle receptacle and the reservoir receptacle. The gap, preferably the air gap (28), is formed in particular by the opening in the intermediate element.

Alternatively or additionally to the embodiment of the intermediate element for forming the air gap (28), the intermediate element is designed in particular as a physical barrier between a microneedle array received in the microneedle receptacle and a reservoir received in the reservoir receptacle. In this embodiment of the intermediate element, this intermediate element is configured in particular to be removable and/or breakable to overcome the contact barrier, preferably the gap. This intermediate element therefore has a predetermined breaking point and/or porosity, for example.

In a preferred embodiment, the application device has a form-fit and/or a force-fit connector for, in particular latching, form-fit and/or force-fit reception of a microneedle array in the microneedle receptacle and/or a reservoir in the reservoir receptacle. Preferably, the microneedle array has a form-fit and/or a force-fit connector for, in particular latching, form-fit and/or force-fit reception of a microneedle array in the microneedle receptacle. Alternatively or additionally, the reservoir receptacle in particular has a form-fit and/or force-fit connector for, in particular latching, form-fit and/or force-fit reception of a reservoir in the reservoir receptacle. It is preferable that the at least one, in particular all, form-fit and/or force-fit connectors are integrally formed with the housing.

In a preferred embodiment, the at least one, in particular all, form-fit and/or force-fit connectors comprise: a groove, in particular for forming a tongue-and-groove connection; an undercut; a projection; a retaining arm, in particular a flexible one; a latch; a closure and/or a fitting, in particular for forming a press-fit connection.

In a preferred embodiment, the microneedle receptacle has a particularly round or rectangular negative form of a microneedle array to be received in the microneedle receptacle. Alternatively or additionally, the reservoir receptacle preferably has a particularly round or rectangular negative form of a reservoir to be received in the reservoir receptacle. The at least one negative form, in particular all negative form, are preferably integrally formed, in particular as openings, with the housing.

In a preferred embodiment, the application device comprises a handle for handling, in particular for placing and/or pressing the application device onto an application site. In particular, the handle is a handle plate. Preferably, the handle is configured such that it can be held from above, from below and/or from the side, e.g. by means of thumb and index finger, in particular without interfering with a reservoir and/or a microneedle array.

The system for microneedle application according to the invention is in particular a system for hydrogel microneedle application. The system comprises an application device having one or more features of an application device according to the invention. Furthermore, the system comprises a microneedle array, in particular one free of active ingredient, received in the microneedle receptacle and/or a reservoir, in particular one containing active ingredient, received in the reservoir receptacle. In particular, the microneedle array and the reservoir are arranged opposite each other, preferably on the end face opposite to each other. Preferably, the reservoir is located at the end face relative to the side of the microneedle array from which the microneedles originate and/or point away. Preferably, the reservoir is located opposite the back of the microneedle array. The microneedle array preferably has 100 to 600 microneedles per cm². Preferably, the microneedle array has conical or pyramid-shaped microneedles. The microneedles of the microneedle array preferably have a length of 100 μm to 1000 μm, preferably approx. 600 μm. Preferably, the microneedles of the microneedle array have a root diameter of 50 μm to 500 μm, particularly preferably of approx. 300 μm. In particular, the reservoir is formed as a reservoir that can be referred to as plaster-shaped and patch-shaped. The microneedle array and/or the reservoir particularly has a rectangular or round shape. Preferably, the microneedle array and/or the reservoir has a diameter of 5 to 25 mm, preferably approx. 11 mm. Preferably, the microneedle array and/or the reservoir has a width of 5 to 25 mm, preferably approx. 11 mm. Preferably, the microneedle array has a preferably plate-shaped support element, which is in particular integrally connected to the microneedles.

In a preferred embodiment, the microneedle array is a particularly swelling hydrogel microneedle array. Preferably, the microneedles, and particularly preferably the entire microneedle array, are swelling. The microneedle array comprises, preferably consists of, Gantrez, PVP and/or PVA.

In a preferred embodiment, the reservoir has at least one active ingredient. In particular, the reservoir contains small molecules, also known as small chemical molecules, and/or biologics. In particular, the reservoir is configured to receive larger quantities of active ingredient compared to dissolving or coated microneedle arrays. The reservoir preferably has a dissolving film, in particular a polymer film, preferably consists of such a film.

It is preferable that the microneedle array and the reservoir are configured to adhere to each other. This means in particular that the microneedle array and the reservoir adhere to each other on contact.

The method according to the invention for providing a system for microneedle application is, in particular, a method for providing a system for hydrogel microneedle application. In particular, the method comprises the step of providing a microneedle array and/or the step of providing a reservoir. One step of the method comprises an opposing arrangement of a microneedle array and a reservoir, in particular one containing active ingredient. Preferably, the arrangement is such that the microneedle array and the reservoir are arranged opposite each other, preferably on the end face opposite to each other. Preferably, the arrangement is such that the reservoir is located at the end face relative to the side of the microneedle array from which the microneedles originate and/or point away. Preferably, the arrangement is such that the reservoir is opposite the back of the microneedle array. When the microneedle array and the reservoir are arranged opposite each other, the microneedle array is spaced relative to the reservoir. The spacing is carried out in particular by means of an application device, particularly preferably having one or more features of the application device according to the invention. The spacing is achieved in particular by means of a contact barrier, wherein the contact barrier is preferably formed as an air gap (28) and/or as a physical barrier. It is particularly preferable that the spacing includes an arrangement so that there is a free space between the microneedle array and the reservoir.

In a preferred embodiment, the method comprises the further step, in particular after the opposing arrangement, of overcoming the gap between the microneedle array and the reservoir to produce a microneedle array system containing an active ingredient. In particular, the gap is overcome such that the microneedle array and the reservoir are connected, in particular permanently. Preferably, an adhesive bond is formed between the microneedle array and the reservoir.

In a preferred embodiment, the gap, in particular the air gap (28), is overcome by a, in particular force-actuated, relative movement of the microneedle array relative to the reservoir. The relative movement is preferably achieved by applying a contact pressure, preferably a manual contact pressure by a user, to the reservoir.

As an alternative or in addition to overcoming the gap by the relative movement, the gap is overcome in particular by removing and/or destroying a contact barrier forming the gap.

It is preferably possible that one of the subject matters of the invention, the application device according to the invention, the system according to the invention and/or the method according to the invention is supplemented with one or more features of the other subject matters of the invention or with one or more features described in the context of the other subject matters of the invention.

### BRIEF DESCRIPTION OF THE DRAWINGS

The terms Fig., Figs., Figure, and Figures are used interchangeably in the specification to refer to the corresponding figures in the drawings.

In the following, the invention is described in more detail by means of preferred embodiments with reference to the accompanying drawings.

The Figures show:

FIG. 1 shows a perspective top view of an embodiment of an application device according to the invention,

FIG. 2 shows a sectional view of the application device of FIG. 1 along II,

FIG. 3 shows a detailed sectional view of the application device of FIG. 2 along III,

FIG. 4 shows a top view of the application device of FIG. 1,

FIG. 5 shows a perspective top view of another embodiment of an application device according to the invention,

FIG. 6 shows a sectional view of the application device of FIG. 5 along II,

FIG. 7 shows a detailed sectional view of the application device of FIG. 6 along III,

FIG. 8 shows a top view of the application device of FIG. 5, and

FIG. 9 shows a sectional view of an embodiment of a system for microneedle application according to the invention with the application device of FIG. 5.

### DESCRIPTION OF THE INVENTION

In the Figures, similar or identical components or elements are identified by the same reference numerals. In particular for improved clarity, elements that have preferably already been identified are not provided with reference numerals in all figures.

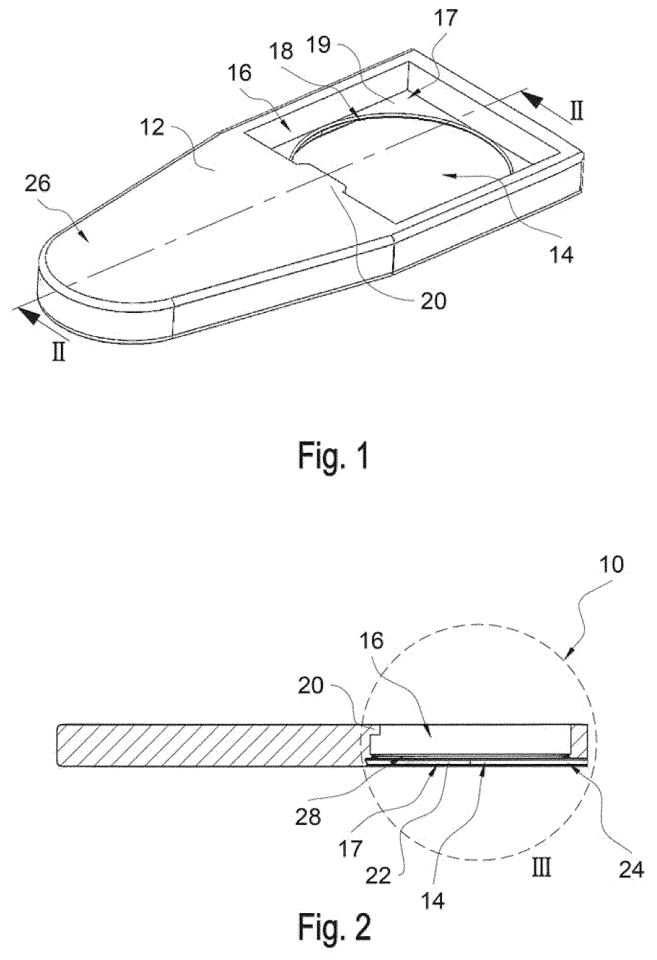

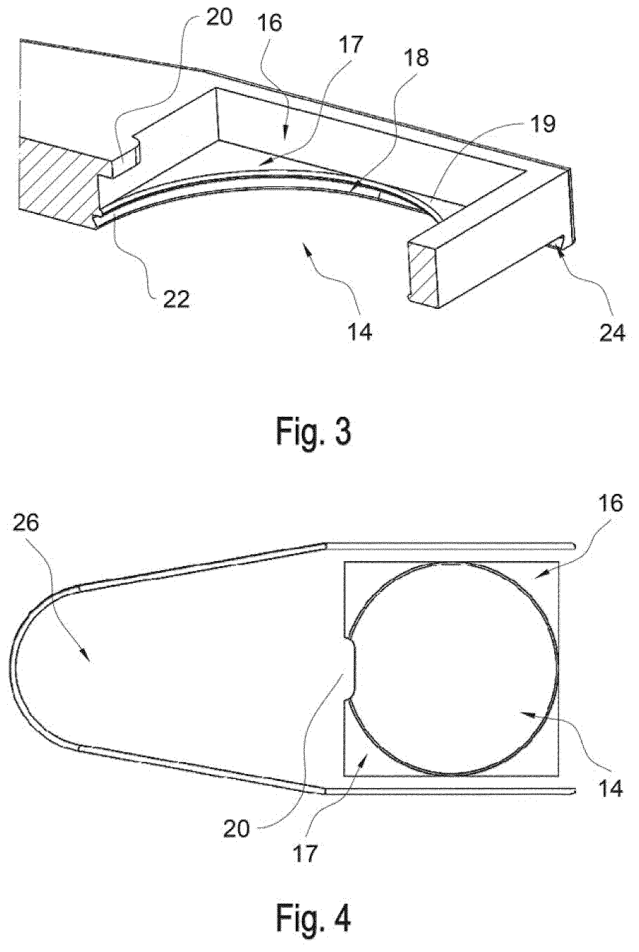

FIG. 1 shows an embodiment of an application device 10 for microneedles according to the invention. FIG. 2 shows a longitudinal sectional view along II of FIG. 1, FIG. 3 shows a detailed view according to III of FIG. 2, and FIG. 4 shows a top view of the embodiment of FIG. 1.

The application device 10 comprises a housing 12 with a handle 26 for handling.

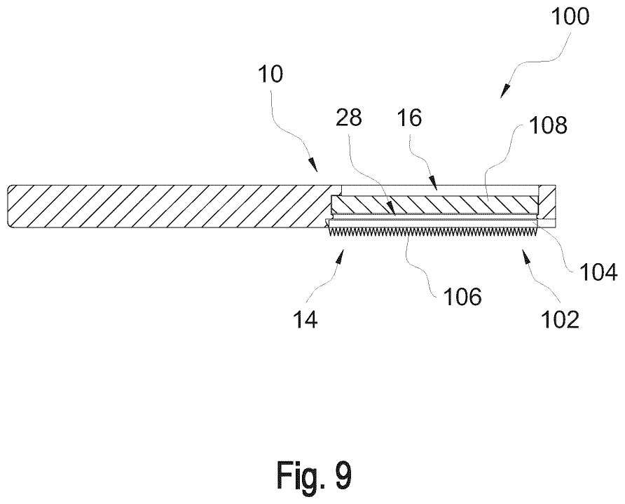

A reservoir receptacle 16 for receiving a reservoir 108 (cf. FIG. 9), in particular one containing active ingredient, is recessed into the housing 12 on the upper side shown in FIG. 1, and a microneedle receptacle 14 for receiving a microneedle array 102 (see FIG. 9), in particular one free of active ingredient, is recessed into the housing 12 on the lower side.

The microneedle receptacle 14 and the reservoir receptacle 16 are formed as openings in the housing 12, wherein in particular the microneedle receptacle 14 forms a negative form for the microneedle array 102 to be received and/or the reservoir receptacle 16 forms a negative form for the reservoir 108 to be received (see FIG. 9).

For in particular form-fit fixing a reservoir 108 (see FIG. 9), the reservoir receptacle 16 comprises an undercut 20, which in particular forms a form-fit and/or a force-fit connector. For in particular form-fit fixing a microneedle array 102 (cf. FIG. 9), the microneedle receptacle 14 comprises a groove 22, which in particular forms a form-fit and/or a force-fit connector. The groove 22, shown on one end face of the application device 10, has an opening 24 for inserting the microneedle array 102. The reservoir receptacle 16 and/or the microneedle receptacle 14 are also preferably configured to be force-fit for fixing the reservoir 108 or the microneedle array 102, for example by means of a fitting. In particular, the application device 10 thus has at least one form-fit and/or force-fit connector such that a reservoir 108 and/or a microneedle array 102 can no longer be removed after insertion and/or is secured against falling out.

The application device 10 comprises a contact barrier 17 for spacing a microneedle array 102 received in the microneedle receptacle 14 and a reservoir 108 received in the reservoir receptacle 16 (see FIG. 9).

The contact barrier 17 is formed by an, in particular a web-shaped, intermediate element 19, having an opening 18. Through the opening 18, an air gap 28 is formed between the microneedle receptacle 14 and the reservoir receptacle 16 or between a microneedle array 102 received in the microneedle receptacle 14 and a reservoir 108 received in the reservoir receptacle 16 for spacing the microneedle array 102 and the reservoir 108.

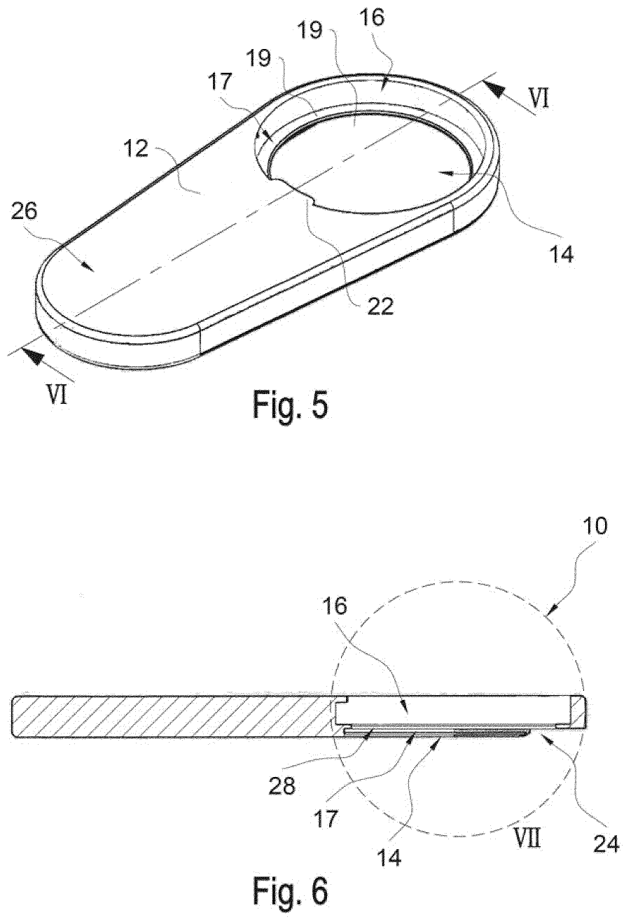

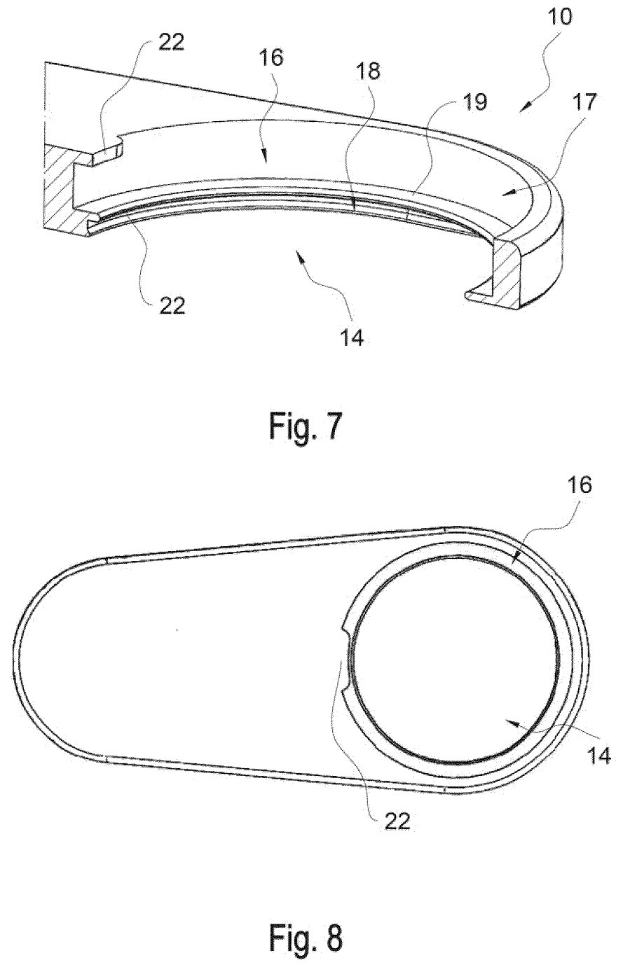

FIGS. 5-8 show another embodiment of an application device 10 for microneedles according to the invention in various views.

The embodiment of FIGS. 5-8 is based on the embodiment of FIGS. 1-4. Instead of the rectangular reservoir receptacle 16 of the embodiment of FIGS. 1-4, the embodiment of FIGS. 5-8 comprises a round reservoir receptacle 16.

FIG. 9 shows an embodiment of a system 100 for microneedle application according to the invention. The system comprises an application device 10 according to the embodiment of FIGS. 5-8.

A reservoir 108, in particular a round one, is fixedly received in the reservoir receptacle 16, the reservoir 108 preferably containing an active ingredient.

A microneedle array 102, in particular one free of active ingredient, is received in the groove 22 of the microneedle receptacle 14. The illustrated microneedle array 102 has a preferably plate-shaped support element 104, in particular integrally connected to the microneedles 106.

The air gap 28 of the contact barrier 17 prevents contact between the microneedle array 102 and the reservoir 108 before an application, so that in particular an ex-change of water and/or solvent is prevented. Advantageously, swelling of the microneedle array 102 prior to application is thus prevented.

During an application, the required force can be applied to the reservoir, causing it to deform downwards and press against the microneedle array 102. The applied force then particularly ensures penetration of the stratum corneum and provides sufficient contact between the two components. This contact is also advantageous so that the active ingredient can diffuse into the skin via the microneedle array 102.

1. An application device for microneedles, in particular hydrogel microneedles, comprising
a microneedle receptacle,
a reservoir receptacle for receiving a reservoir, in particular one containing an active ingredient, and
a contact barrier for spacing a microneedle array received in the microneedle receptacle and a reservoir received in the reservoir receptacle.

2. The application device according to claim 1, wherein the contact barrier is a gap, in particular an air gap

3. The application device according to claim 2, wherein the distance formed by the contact barrier is 0.01 mm to 3 mm, preferably 0.05 mm to 1 mm, particularly preferably 0.1 mm to 0.5 mm.

4. The application device according to claim 1, wherein the contact barrier (17) adapted to be overcome by a, in particular force-actuated, relative movement of a microneedle array received in the microneedle receptacle (14) relative to a reservoir (108) received in the reservoir receptacle (16).

5. The application device according to claim 1, wherein an intermediate element, in particular a spacer, between the microneedle receptacle and the reservoir receptacle for forming the contact barrier.

6. The application device according to claim 5, wherein the intermediate element has an opening between the microneedle receptacle and the reservoir receptacle.

7. The application device -according to claim 5, wherein the intermediate element is removable and/or breakable to overcome the contact barrier.

8. The application device according to claim 1, wherein a form-fit and/or force-fit connector for, in particular latching, form-fit and/or force-fit reception of a microneedle array in the microneedle receptacle and/or of a reservoir in the reservoir receptacle.

9. The application device according to claim 8, the form-fit and/or force-fit connector comprise: a groove; an undercut; a projection; a retaining arm, in particular a flexible one; a latch; a closure and/or a fitting.

10. The application device according to claim 1, wherein the microneedle receptacle has a, in particular round or rectangular, negative form of a microneedle array to be received in the microneedle receptacle, and/or the reservoir receptacle has a, in particular round or rectangular, negative form of a reservoir to be received in the reservoir receptacle.

11. The application device according to claim 1, wherein a handle for handling, in particular placing and/or pressing the application device onto an application site.

12. A system for microneedle application, in particular hydrogel microneedle application, comprising
an application device according to claim 1,
a microneedle array received in the microneedle receptacle, in particular one free of active ingredient, and/or
a reservoir received in the reservoir receptacle, in particular one containing an active ingredient.

13. The system according to claim 12, wherein the microneedle array is a, in particular swelling, hydrogel microneedle array.

14. The system according to claim 12, wherein the microneedle array and the reservoir are configured to be adhesive to one another.

15. A method for providing a system for microneedle application, in particular a system for hydrogel microneedle application, comprising the step of opposingly arranging a microneedle array and a reservoir, in particular one containing an active ingredient, wherein during the opposing arrangement the microneedle array is spaced relative to the reservoir, preferably by means of an application device-particularly preferably by means of an application device according to claim 1.

16. The method according to claim 15, wherein the further step of overcoming the distance between the microneedle array and the reservoir to produce a microneedle array system containing an active ingredient.

17. The method according to claim 16, wherein the distance is overcome by a, in particular force-actuated, relative movement of the microneedle array relative to the reservoir.

18. The method according to claim 16, wherein the distance is overcome by removing and/or destroying a contact barrier forming the distance.
